(19) United States
(12) Patent Application Publication
(10) Pub. No.: US 2022/0347096 A1
(43) Pub. Date: Nov. 3, 2022

(54) LIPOSOMAL CANNABINOIDS AND USES THEREOF

(71) Applicant: YISSUM RESEARCH DEVELOPMENT COMPANY OF THE HEBREW UNIVERSITY OF JERUSALEM LTD, Jerusalem (IL)

(72) Inventors: Yechezkel BARENHOLZ, Jerusalem (IL); Ahuva CERN, Modiin (IL)

(21) Appl. No.: 17/765,625

(22) PCT Filed: Oct. 1, 2020
(86) PCT No.: PCT/IL2020/051068

§ 371 Date: Mar. 31, 2022

Related U.S. Application Data
(60) Provisional application No. 62/910,097, filed on Oct. 3, 2019.

(51) Int. Cl. A61K 9/127; A61K 47/40; A61K 31/352; A61K 47/42

(52) U.S. Cl. CPC A61K 9/127; A61K 47/40; A61K 31/352; A61K 47/42; A61K 9/0019

(57) ABSTRACT

The present disclosure provides prolonged release formulation of cannabinoids. The formulation comprises liposomes having a lipid membrane and an intraliposomal aqueous core, wherein said liposome comprises either an entrapped cannabinoid and at least one dispersing agent of said cannabinoid, said dispersing agent being other than a cyclodextrin (CD) compound; or an entrapped cannabinoid, at least a portion of said cannabinoid being entrapped in said lipid membrane, and wherein said lipid membrane comprises a mole ratio between said cannabinoid and said one or more liposome forming lipids in the range of 1 to 10. Also disclosed are methods of preparing and uses of the formulations for prolonged delivery of the cannabinoids and therapeutic treatments making use of same.

### TECHNOLOGICAL FIELD

The present disclosure concerns liposomal cannabinoids.

### BACKGROUND ART

References considered to be relevant as background to the presently disclosed subject matter are listed below:
international patent application publication No, WO2017203529
international patent application publication No. WO2001003668
US patent application publication No. US20170044092
International patent application publication No. WO2018145213
US patent application publication No. US20180193399
U.S. Pat. No. 9,095,555
U.S. Pat. No. 1,011,7883
US patent application publication No. US20170281701
US patent application publication No. US20180318237
US patent application publication No. US20180271924A1
US patent application publication No. US20170107280
international patent application publication No. WO2017191630
US patent application publication No. US20180303791
US patent application publication No. US20180042845
US patent application publication No. US20180185324
US patent application publication No. US20180289665
U.S. Pat. No. 9,655,910
U.S. Pat. No. 8,242,178
US patent application publication No. US20180221304 Acknowledgement of the above references herein is not to be interred as meaning that these are in any way relevant to the patentability of the presently disclosed subject matter.

### BACKGROUND

There are few publications describe the use of CBD associated with various liposomes, for example: WO2017203529 describes compositions comprising a combination of cannabidiol (CBD) or a derivative thereof, and hyaluronic acid or a salt thereof; a phospholipid, and optionally a physiologically acceptable carrier. The CBD may be incorporated into liposomes formed by the phospholipids. The composition is described for use in treating inflammatory joint diseases, or pain or inflammation associated with such diseases. The composition is formulated for local injection.

Other publications that describe liposomal CBD include WO2001003668 describing pulmonary delivery of liposome-encapsulated cannabinoids; US20180318237 describing topical administration of cannabinoids, possibly within liposomes; WO2017191630 describing the use of cannabidiol, possibly in liposomes, for reducing a steroid dose and treating inflammatory and autoimmune diseases; US20180303791, US20180042845 and US20180185324 describing the use of cannabinoids for treating multiple myeloma, the cannabinoids may be within liposomes; U.S. Pat. No. 9,655,910 describing the use of cannabinoids, possibly in liposomes, for treating addiction; U.S. Pat. No. 8,242,178 describing the use of cannabidiol, possibly in liposomes, for treating autoimmune hepatitis; and US20180221304 describing cannabinoid-containing complex mixtures for the treatment of mast cell-associated or basophil-mediated inflammatory disorders, with liposomes being suggested as a tool for topical delivery.

### GENERAL DESCRIPTION

The present disclosure provides a prolonged release formulation comprising liposomes having a lipid membrane and an intraliposomal aqueous core, wherein the liposome comprises one or more liposome forming lipids, an entrapped cannabinoid (e.g. cannabidiol (CBD)) or functional homologue thereof and at least one dispersing agent of a cannabinoid (e.g. PG, HSA, IVIg) that is not a cyclodextrin (CD) compound. The intraliposomal core may include also CD.

Also provided by the present disclosure is a prolonged release formulation comprising liposomes having a lipid membrane and an intraliposomal aqueous core, the lipid membrane comprises one or more liposome forming lipids, wherein said liposome comprises an entrapped cannabinoid, at least a portion of said cannabinoid being entrapped in said lipid membrane, and wherein said lipid membrane comprises a mole ratio between said cannabinoid and said one or more liposome forming lipids in the range between 1 to 10.

Also provided by the present disclosure is a method of treatment comprising administering a subject in need a therapeutically effective amount of a prolonged release formulation comprising liposomes having a lipid membrane and an intraliposomal aqueous core, wherein the liposome comprises one or more liposome forming lipids, an entrapped cannabinoid compound and at least one dispersing agent of a cannabinoid that is not a CD compound.

Yet further, provided by the present disclosure is a method of treatment comprising administering to a subject in need of treatment a prolonged release formulation comprising liposomes having a lipid membrane and an intraliposomal aqueous core, the lipid membrane comprises one or more liposome forming lipids, wherein said liposome comprises an entrapped cannabinoid, at least a portion of said cannabinoid being entrapped in said lipid membrane, and wherein said lipid membrane comprises a mole ratio between said cannabinoid and said one or more liposome forming lipids in the range between 1 to 10.

In some examples, the formulation also comprises an entrapped CD compound.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIGS. 1A-1C are microscopic images (Zeiss SN 221209, ×200 magnification) of various CBD formulations including liposomal CBD (F1, FIG. 1A), Liposomal CBD-HSA 50 mg/ml; (FIG. 1B), Liposomal CBD-HSA 100 mg/ml (FIG. 1C).

FIGS. 2A-2B are graphs showing plasma CBD concentrations (ng/ml) after IV administration of 12 mg/kg dose vs the different IM formulations (n=3, mean±SD, w/o outlier in two separate in vivo studies.

FIGS. 3A-3B are graphs showing absolute CBD released (mg) from the muscles after IM administration of the different formulations (n=3, mean±SD, w/o outlier) (FIG. 3A) and Percent CBD released from the muscles after IM administration of the different formulations (n=3, mean±SD, w/o outlier) (FIG. 3B).

FIG. 4 is a microscopic image (Zeiss SN 221209. ×200 magnification) of DMPC:DPPC-CBD liposomes

### DETAILED DESCRIPTION

The present disclosure is based on the unexpected finding that the presence of CBD entrapped in a liposome bilayer affects (reduces/slows) the release rate of CBD from the liposome, allowing for a prolonged delivery of CBD from the liposomal formulation. The present disclosure also finds basis in the finding that the presence of a dispersing agent (capable of homogenously dispensing CBD) in the intraliposomal aqueous environment (with or without CBD in the intraliposomal aqueous environment) can also reduce the release rate of CBD from the liposome.

Thus, in accordance with a first of its aspects the present disclosure provides a prolonged release formulation comprising liposomes having a lipid membrane and an intraliposomal aqueous core, wherein the liposome comprises one or more liposome forming lipids, an entrapped cannabinoid compound and at least one cannabinoid dispersing agent other than (i.e. that is not) a cyclodextrin (CD) compound.

In accordance with a second aspect, the present disclosure provides a prolonged release formulation comprising liposomes having a lipid membrane and an intraliposomal aqueous core, the lipid membrane comprises one or more liposome forming lipids, wherein said liposome comprises an entrapped cannabinoid or functional homologue thereof, at least a portion of said cannabinoid being entrapped in said lipid membrane, and wherein said lipid membrane comprises a mole ratio between said cannabinoid and said one or more liposome forming lipids in the range between 1 to 10.

Also provided by the present disclosure is a method making use of the aforementioned prolonged liposomal formulation for treating a condition in need of prolonged delivery of a cannabinoid compound, the method thus comprising administration of the prolonged release formulation to said subject.

The formulations disclosed herein comprise at least one cannabinoid. In the context of the present disclosure, when referring to cannabinoid, it is to be understood as encompassing a single compound or a combination of cannabinoid compounds (i.e. the term as used herein encompasses a single or a plurality of such compounds). In some examples, the combination of cannabinoids comprises components of the plant extract, i.e. multiple cannabinoids and optionally plant flavonoids and terpenoids.

In some examples, the cannabinoid is or comprises cannabidiol (CBD).

In some other examples, the cannabinoid is or comprises tetrahydrocannabinol (THC) (Delta9-THC and/or Delta8-THC).

Other cannabinoids that fall within the scope of the present disclosure include one or any combination of two or more cannabinoids selected from the group consisting of cannabigerol (CBG), cannabigerolic acid (CBGA), cannabigerol monomethyl ether (CBGM), cannabichromene (CBC), cannabichromanone (CBCN), cannabichromenic acid (CBCA), cannabivarichromene (CBCV), cannabichromevarinic acid (CBCVA), isotetrahydrocannabinol (iso-THC), cannabinol (CBN), cannabinolic acid (CBNA), cannabinol methyl ether (CBNM), cannabinol C₄ (CBN-C₄), cannabinol C₂ (CBN-C₂), cannabinol C₁ (CBN-C₁), cannabinodiol (CBND), cannabielsoin (CBE), cannabielsoic acid A (CBEA-A), Cannabielsoic acid B (CBEA-B), cannabicyclol (CBL), cannabicycloic acid (CBLA), cannabicyclovarin (CBLV), cannabitriol (CBT), cannabitriolvarin (CBTV), ethoxy-cannabitriolvarin (CBTVE), cannabivarin (CBV), cannabinodivarin (CBVD), tetrahydrocannabivarin (THCV), cannabidivarin (CBDV), cannabigerovarin (CBGV), cannabigerovarinic acid (CBGVA), cannabifuran (CBF), dehydrocannabifuran (DCBF), cannabirispol (CBR), each constituting a separate embodiment of the present disclosure.

In some examples, the cannabinoid is or comprises a combination of CBD and any one or more of the above listed cannabinoids.

In some preferred examples, the cannabinoid within the formulation is CBD.

The term CBD compound encompasses, in the context of the present disclosure CBD as well as functional homologues thereof. When referring to a CBD functional homologue it is to be understood as a compound having similar physico-chemical properties as CBD.

In some examples, the CBD functional homologue is a chemical analogue of CBD containing at least one benzene ring and a log P above 4.

In some examples, a CBD functional homologue includes structural homologue (including isomers) of CBD that, similar to CBD is lacking the psychoactivity of Tetrahydrocannabinol (THC).

In some examples, the CBD compound is a natural phytocannabinoids.

In some examples, the CBD compound is a synthetic CBD homologue.

Non-limiting examples of CBD compounds include name 2-[(1R,6R)-6-Isopropenyl-3-methylcyclohex-2-en-1-yl]-5-pentylbenzene-1,3-diol (CBD), the synthetic Cannabidiol-dimethylheptyl (CBD-DNH), the phytocannabinoids Cannabidivarin (CBDV), Cannabidivarinolic acid (CBDVA), Cannabidiol monomethyl ether (CBDM) [Paula Morales, Patricia H. Reggio, and Nadine Jagerovic “An Overview on Medicinal Chemistry of Synthetic and Natural Derivatives of Cannabidiol” Front Pharmacol.” 8:422, (2017)].

In some examples, the active ingredient is CBD known by its chemical name 2-[(1R,6R)-6-Isopropenyl-3-methylcyclohex-2-en-1-yl]-5-pentylbenzene-1,3-diol.

The cannabinoid, and preferably CBD compound is entrapped in/associated with the liposomes. In the context of the present disclosure, when referring to entrapment of the compound in the liposome it is to be understood as defining any form of physical or chemical association between the cannabinoid and the liposome per se. Yet, it should be clear that the physical association in only due to the phospholipids being in a form of a liposomes and that there is no chemical association between the cannabinoid and phospholipid per se. The association may be by having the cannabinoid enclosed within the intraliposomal aqueous core/medium, and/or at least partially embedded within the lipid membrane (e.g. due to the hydrophobicity of the cannabinoid), and/or associated with the outer surface of the liposome (e.g. by physical forces).

The amount of cannabinoid entrapped in the liposomes can be determined using commercial chromatography techniques. In some examples, the concentration of cannabinoid is determined using a High-Performance Liquid Chromatography (HPLC)/UV method

In some examples, the cannabinoid entrapped in the lipid membrane is determined by methods known in the art. For example, and without being limited thereto, the ratio between the one or more liposome forming lipids in the lipid membrane and the CBD can be determined by differential scanning calorimetry (DSC).

To calculate the intra-liposomal concentration of cannabinoid one also need the aqueous intraliposome trapped volume which can be calculated as described previously [Bangham A D, et. al. (1965) J Mol Biol. 13(1):238-52]

In some examples, the amount of cannabinoid and preferably the CBD compound entrapped by the liposome is at least 30 mg/ml; at times, at least 40 mg/ml; at times, at least 50 mg/ml; at times, at least 60 mg/ml; at times, at least 70 mg/ml; at times, at least 80 mg/ml; at times, at least 90 mg/ml; at times, at least 100 mg/ml; at times, at least 110 mg/ml; at times, at least 120 mg/ml; at times, at least 130 mg/ml; at times, at least 140 mg/ml; at times, at least 150 mg/ml; at times, at least 160 mg/ml; at times, at least 170 mg/ml; at times, at least 180 mg/ml; at times, at least 190 mg/ml; and even at least 20 mg/ml.

In some examples, the amount of the cannabinoid and preferably the CBD compound entrapped by the liposome is at most 400 mg/ml; at times, at most 350 mg/ml; at times, at most 330 mg/ml; at times, at most 310 mg/ml; at times, at most 300 mg/ml; at times, at most 280 mg/ml; at times, at most 260 mg/ml; at times, at most 240 mg/ml; at times, at most 220 mg/ml; at times, at most 200 mg/ml; at times, at most 190 mg/ml; at times, at most 180 mg/ml; at times, at most 170 mg/ml; at times, at most 160 mg/ml; at times, at most 150 mg/ml; at times, at most 140 mg/ml; at times, at most 130 mg/ml; at times, at most 120 mg/ml.

In some examples, amount of cannabinoid and preferably the CBD compound entrapped by the liposome is in the range of 30-400 mg/ml, at times, in the range of 30-350 mg/ml; at times, in the range of 30-350 mg/ml; at times, in the range of 30-350 mg/ml; at times, in the range of 30-350 mg/ml; at times, in the range of 30-200 mg/ml; at times, in the range of 50-250 mg/ml; at times, in the range of 40-180 mg/ml; at times, in the range of 40-250 mg/ml; at times, in the range of 30-120 mg/ml; at times, in the range of 40-150 mg/ml; at times, in the range of 50-300 mg/ml; or in any range within the above identified lower and upper concentration limits.

In some examples, the cannabinoid to lipid mole ratio is determined.

In some examples, the cannabinoid compound/lipid mole ratio is between 1 to 10, at times, between 1 to 9, at times, between 1 to 8, at time between 1 to 7, at times between 1 to 6, at times between 1 to 5.

A unique feature of the present disclosure resides in the presence, in the intraliposomal compartment, of a cannabinoid and preferably CBD compound in combination with at least one cannabinoid dispersing agent that is not cyclodextrin (CD). This is unique due to the very low solubility of cannabinoids, such as CBD in aqueous solution (CBD having predicted log P of 7.03) that is achieved using the different dispersing agents. Without being bound by theory, it is considered that the dispersing agent (that is not a CD compound, yet may be combined with a CD compound), when present in the liposome in the intraliposomal aqueous core maintains an amount of the cannabinoids in dissolved or homogenously dispersed form and thereby improves the sustainability of the cannabinoids within the liposome.

In the context of the present disclosure, the term “cannabinoid dispersing agent” should be understood to encompass any chemical entity that facilitates or enhances the dispersibility of cannabinoids (one or combination of cannabinoids) in the liquid medium used for loading of the cannabinoid(s) into the liposomes (preferably, although not exclusively, by passive loading). without being bound by theory, the cannabinoid dispersing agent physically associates with the cannabinoid and thereby becomes entrapped within the liposomes, in the form of non-covalent complexes.

In some examples, the dispersing agent is a solubilizer (also recognized by the term solubilization enhancing agent). When referring to the solubilizer it is to be understood to encompass at least one compound that is not CD. Thus, in the context of the present disclosure the term “solubilizer other than CD” should be understood as any solubilization enhancing compound that is not CD, and yet it may be combined with CD, as an additional solubilization enhancing compound.

Solubilizers are known to be used for improving drug solubility, particularly when using insoluble drugs or those having low solubility. In some examples of the present disclosure, cannabinoid is added both in the lipid and aqueous phases and therefore it is believed that the cannabinoid compound is distributed between the lipid phase (lipid membrane) and the aqueous intraliposomal phase, thus providing two different pools for the active ingredient (i.e. cannabinoid, such as CBD compound).

In other words, and without being bound by theory, it is believed that the dispersing agent, maintain cannabinoid in the intraliposomal aqueous medium and facilitate in the controlled (specifically, prolonged, e.g. for even up to 3 weeks) release of the active ingredient, e.g. CBD compound, from the liposome.

There are different types of solubilizers. Solubilizers may be co-solvents, namely, substances that are added to a primary solvent (be it an organic solvent or water) in small amounts to increase/improve solubility of poorly soluble compounds; such as, and without being limited thereto, polyethyleneglycol (PEG), e.g. PEG300, PEG400, propylene glycol (PG), N,N-dimethylacetamide (DMA), ethanol; or they may be recognized as surfactants such as, and without being limited thereto, Tween80 (Polyoxyethylene (20) sorbitan monooleate), Cremophor (propane-1,2,3-triol:oxirane (1:1)) or they may be recognized as complexing agents such as members of the cyclodextrin family of compounds.

In some examples, the solubilizer is a co-solvent. A preferred co-solvent is PEG. Another preferred co-solvent is PG.

The dispersing agent can be other than a solubilizer. In some examples, the dispersing agent is a protein that is selected by its capability to disperse cannabinoids, and preferably CBD, in the aqueous medium in which it is dissolved.

In some examples, the dispersing protein is a serum protein.

In some examples, the serum protein is albumin.

In some examples, the serum protein is human serum albumin (HSA).

In some examples, the serum protein is a globulin.

In some examples, the serum protein is an immunoglobulin.

The dispersing agent and the cannabinoid are associated by non-covalent linkage. In some examples, the dispersing agent and the cannabinoid form a physical complex, such that, under suitable conditions, allow the release of the cannabinoid from the dispensing agent. Thus, the dispersing agent and the cannabinoid are, in some examples, non-covalently bound one to each other.

In some examples, the formulation includes a combination of two or more dispersing agents.

In some examples, the combination of two or more dispersing agents include at least a cyclodextrin (CD) compound.

In some examples, the combination of dispersing agents includes two or more such compounds, none of which is a CD compound.

As noted above, the liposomes may include also a CD compound. CD compounds are recognized as cyclic oligosaccharides consisting of (α-1,4)-linked α-D-glucopyranose units and contain a lipophilic central cavity and hydrophilic outer surface. In the context of the present disclosure, the CD can be a naturally occurring CD, as well as derivatives of the naturally occurring CDs. Natural CD include the α-, β-, or γ-cyclodextrin (αCD, βCD or γCD) consisting of six, seven and eight glucopyranose units, respectively. When referring to derivatives of the natural CD (which are also encompassed under the general term “CD compound”) it is to be understood as any cyclic oligosaccharides consisting of (α-1,4)-linked α-D-glucopyranose units having a lipophilic central cavity and hydrophilic outer surface.

In some examples, the CD compound is 2-hydroxypropyl-β-cyclodextrin (HPβCD).

In some examples, the CD compound is 2-hydroxypropyl-γ-cyclodextrin (HPγCD).

In some examples, the CD compound is Solfobutyl ether (SBE) cyclodextrin.

In one preferred example, the CD is HPβCD or in short HPCD.

The formulation comprises also the liposomes.

The liposomes are prepared a priori with at least one liposome forming lipid. In the context of the present invention, the term “liposome forming lipids” denotes primarily glycerophospholipids or sphingomyelins that form in water into vesicles, such as, but without being limited thereto, liposomes, as further discussed below.

When referring to glycerophospholipids it is to be understood as lipids having a glycerol backbone wherein at least one, preferably two, of the hydroxyl groups at the head group is substituted by one or two of an acyl, alkyl or alkenyl chain, a phosphate group, or combination of any of the above, and/or derivatives of same and may contain a chemically reactive group (such as an amine, acid, ester, aldehyde or alcohol) at the head group, thereby providing the lipid with a polar head group. The sphingomyelins consist of a ceramide unit with a phosphorylcholine moiety attached to position 1 and thus in fact is an N-acyl sphingosine. The phosphocholine moiety in sphingomyelin contributes the polar head group of the sphingomyelin.

In the liposome forming lipids the acyl, alkyl or alkenyl chain is typically between 12 to about 24 carbon atoms in length, and have varying degrees of saturation being fully, partially or non-hydrogenated naturally occurring lipids, semi-synthetic or fully synthetic lipids and the level of saturation may affect rigidity of the liposome thus formed (typically lipids with saturated chains are more rigid than lipids of same chain length in which there are un-saturated chains, especially having cis double bonds).

In some examples, the liposome comprises a single type of liposome forming lipids.

In some other examples, the liposome comprises a combination of liposome forming lipids.

In some examples, the liposome forming lipid is a phospholipid. When the liposome forming lipid is phospholipid, the amount thereof in the liposome can be determined as organic phosphorous by the modified Bartlett method [Shmeeda H, Even-Chen S, Honen R, Cohen R. Weintraub C, Barenholz Y. 2003. Enzymatic assays for quality control and pharmacokinetics of liposome formulations: comparison with nonenzymatic conventional methodologies. Methods Enzymol 367:272-92]. The lipids can also be tested using the ELSD/HPLC method described herein, in the non-limiting Examples, which form an integral part of the present disclosure.

In some examples, the liposome forming lipid is a choline-type phospholipids such as diacylglycero-phosphocholine (the acyl, alkyl or alkenyl chain being as defined above).

In some other examples, liposome forming lipid is di-lauroyl-sn-glycero-2phosphocholine (DLPC). In some examples, liposome forming lipid is 1,2-dimyristoyl-sn-glycero-3-phosphocholine (DMPC). In some examples, liposome forming lipid is 1,2-dipalmitoyl-sn-glycero-3-phosphocholine (DPPC). In some examples, the liposome forming lipid is 1,2-dipalmitoyl-sn-glycero-3-phosphocholine (DPPC). In some examples, the liposome forming lipid is 1,2-diheptadecanovl-sn-glycero-3-phosphocholine. In some examples, the liposome forming lipid is 1,2-distearoyl-sn-glycero-3-phosphocholine (DSPC). In some examples, the liposome forming lipid is 1,2-dinonadecanoyl-sn-glycero-3-phosphocholine. In some examples, the liposome forming lipid is 1,2-diarachidoyl-sn-glycero-3-phosphocholine (DBPC). In some examples, the liposome forming lipid is 1,2-dihenarachidoyl-sn-glycero-3-phosphocholine. In some examples, the liposome forming lipid is 1,2-dibehenoyl-sn-glycero-3-phosphocholine 1,2-ditricosanoyl-sn-glycero-3-phosphocholine. In some examples, the liposome forming lipid is 1,2-dilignoceroyl-sn-glycero-3-phosphocholine. In some examples, the liposome forming lipid is 1-myristoyl-2-stearoyl-sn-glycero-3-phosphocholine. In some examples, the liposome forming lipid is 1-palmitoyl-2-stearoyl-sn-glycero-3-phosphocholine (PSPC). In some examples, the liposome forming lipid is 1-stearoyl-2-palmitoyl-sn-glycero-3-phosphocholine (SPPC). In some examples, the liposome forming lipid is 1,2-di-oleoyl-sn-glycero-3-phosphocholine (DOPC) or di-lauroyl-sn-glycero-2phosphocholine (DLPC).

In some examples, the liposome forming lipid comprises at least hydrogenated soy phosphatidylcholine (HSPC).

In one preferred example, the liposome forming lipid comprises or consists of hydrogenated soy phosphatidylcholine (HSPC).

In some examples, the liposome forming lipid comprises at least 1,2-dipalmitoyl-sn-glycero-3-phosphocholine (DPPC).

In some examples, the liposome forming lipid comprises at least 1,2-dimynristoyl-sn-glycero-3-phosphocholine (DMPC).

In one preferred example, the liposome forming lipid comprises or consists of a combination of DMPC and DPPC. In some example, the two liposome forming lipids are at a DMPC:DPPC mole ratio of about 45:55. Without being limited thereto, the DMPC:DPPC containing formulation is suitable non-human (veterinary) use.

In some examples the liposome comprises a sterol, such as cholesterol.

In some examples, when cholesterol is present in the liposome, it is in amount of not more than 4% molar.

In some additional or other examples, the liposomes comprise a lipopolymer, such as polyethylene glycol derived lipids (PEGylated lipids).

The liposomes can be of any form or size.

In some examples, the liposomes are multilamellar or oligolamellar vesicles.

In some examples, the liposomes are multivesicular vesicles.

In some other examples, the liposomes are unilamellar vesicles, preferably large unilamellar vesicles.

The liposomes can be small, medium, large or even giant. When referring to small liposomes it is to be understood as having an average size in the range of between about 20 nm-100 nm; when referring to medium sized liposomes, it is to be understood as having an average size in the range of between about 100 nm-200 nm; when referring to large liposomes, it is to be understood as having an average size above about 200 nm; and when referring to giant liposomes (typically giant unilamellar or multivesicular vesicles), it is to be understood as referring to those being larger than 1 μm.

In some examples, the liposomes are multilamellar vesicles (MLV). In some examples, the MLV have a size distribution with a minimum equal or above 100 nm.

In some examples, the formulation comprising the liposomes is in dry form. Specifically, although not exclusively, the liposomes are lyophilized.

In some other examples, the formulation comprises the liposomes being held within a medium, referred to herein as by the term “external medium”. The external medium may be of any composition suitable for holding therein the liposomes. In some examples, the external medium is one suitable for storage of the liposomes, and in some other examples, the external medium is one suitable for administration of the liposomes, e.g. a physiologically acceptable carrier.

In some examples, typically when the external medium is one suitable for administration, the external medium may include a cannabinoid. The cannabinoid may be the same or different from the cannabinoid entrapped by the liposome.

The combination of the cannabinoid and the dispersing agent allows for the formation of the prolonged release formulation, preferably, in controlled manner. In the context of the present disclosure, when referring to “controlled release” or “prolonged release” it is to be understood as meaning a controlled release over a period of time. The period of time includes at least several days, at times at least 3 days; at times, at least 4 days; at times, at least 5 days; at times, at least 6 days; at times, at least 7 days; at times, at least 8 days; at times, at least 9 days; at times, at least 10 days; at times, at least 11 days; at times, at least 12 days; at times, at least 13 days; at times, at least 14 days; at times, at least 15 days; at times, at least 16 days; at times, at least 17 days; at times, at least 18 days; at times, at least 19 days; at times, at least 20 days; at times, at least 21 days, or even more than 30 days. The term “prolonged release” encompasses any form of controlled release other than immediate release (e.g. where more than 50% is released within the first 24 hours) and includes extended/prolonged release and/or delayed release. The prolonged release can be determined by in vitro release assay as described in to Example 3. Release of ≤70%, at times, ≤60%, at times ≤50%, in 50% serum after 2 h of incubation may be regarded as prolonged release.

The present disclosure also provides in the formulation a physiologically acceptable carrier suitable for administration by injection or infusion.

In the context of the present invention, a physiologically acceptable carrier is denotes any carrier that is useful in preparing a pharmaceutical formulation that is generally safe, non-toxic and neither biologically nor otherwise undesirable. In some examples, the physiologically acceptable carrier is an aqueous based solution suitable for administration by injection. In some examples, the physiologically acceptable carrier suitable for systemic administration include aqueous and non-aqueous, isotonic sterile injection/infusion solutions, which can contain anti-oxidants, buffers, bacteriostats, and solutes that render the formulation isotonic with the blood of the intended recipient. In some examples, the carrier is any one or combination of saline, buffered solution, aqueous sugar solutions (dextrose, sucrose etc), etc. In some examples, the carrier may also include thickening agents, stabilizers, and preservatives.

In some examples, the administration is by any one of intramuscular (i.m.), intra-peritoneal (i.p.), intravenous (i.v.), and subcutaneous (s.c.) injection

In one preferred example, the liposomal formulation is for IM injection. The IM injection exhibited a prolonged/extended release profile providing an advantage over IV injection of non-liposomal cannabinoid formulations.

In some examples, the administration to a mammalian subject.

In some examples, the administration is to a human subject.

In some other examples, the administration is to a non-human (i.e. veterinary) subject.

The amount of the cannabinoid compounds in the liposomes is designed to be sufficient to provide a therapeutic effect upon administration of the formulation to a subject.

An amount sufficient or effective to achieve a therapeutic effect upon administration is to be understood as including at least one therapeutic effect known to be achieved by or associated with cannabinoid compounds, particularly with CBD.

Without being limited thereto the therapeutic effect can be in any one or combination of treating/ameliorant/reducing pain and/or inflammation, as well as any other therapeutic effect known to be associated with the administration of the particular cannabinoid compounds, particularly CBD.

The amount of cannabinoids to be delivered by the disclosed liposomal formulation depends on various parameters as known to those skilled in the art and can be determined based on appropriately designed clinical trials (dose range studies) and the person versed in the art will know how to properly conduct such trials in order to determine the effective amount. The amount depends, inter alia, on the type and severity of the disease to be treated and the treatment regime (mode of administration), gender and/or age and/or weight of the treated subject, etc.

The liposomes in the formulation and the formulations per se may be characterized by any technique or any parameter known in the art of liposomal formulations. This includes, without being limited thereto, liposome size and/or size distribution (e.g. using dynamic light-scattering (DLS)), polydispersity index (PDI), zeta potential, measurements of dispersion pH using pH meter, etc.

The present disclosure also provides a method for the administration of treating a subject with cannabinoids the method comprises administration to a subject in need of such treatment of the liposomal formulation disclosed herein.

In view of the above, in the context of the present disclosure, when referring to treatment by the formulation or liposomes disclosed herein, it is to be understood as encompassing ameliorating undesired symptoms associated with a disease, preventing the manifestation of such symptoms before they occur, slowing down the progression of a disease, slowing down the deterioration of symptoms, enhancing the onset of remission period of a disease, slowing down irreversible damage caused in progressive chronic stages of a disease, delaying onset of progressive stages, lessening severity or cure a disease, improving survival rate or more rapid recovery from a disease, preventing the disease from occurring, or a combination of two or more of the above.

As used herein, the forms “a”, “an” and “the” include singular as well as plural references unless the context clearly dictates otherwise. For example, the term “cannabinoid” includes one or more cannabinoids.

Further, as used herein, the term “comprising” is intended to mean that the liposome include cannabinoids and the dispensing agent, but not excluding other elements, such as physiologically acceptable carriers and excipients as well as other agents. The term “consisting essentially of” is used to define, for example, liposomes which include the recited elements but exclude other elements that may have an essential significance on the delivery of cannabinoids. “Consisting of” shall thus mean excluding more than trace elements of such other elements. Embodiments defined by each of these transition terms are within the scope of this invention.

Further, all numerical values, e.g. when referring the amounts or ranges of the elements constituting the liposomes and formulations comprising the same are approximations which are varied (+) or (−) by up to 20%, at times by up to 10% of from the stated values. It is to be understood, even if not always explicitly stated that all numerical designations are preceded by the term “about”.

The invention will now be described by way of non-limiting examples that were carried out in accordance with the invention. It is to be understood that these examples are intended to be in the nature of illustration rather than of limitation. Obviously, many modifications and variations of these examples are possible in light of the above teaching. It is therefore, to be understood that within the scope of the appended claims, the invention may be practiced otherwise, in a myriad of possible ways, than as specifically described hereinbelow.

### DESCRIPTION OF EMBODIMENTS

### Example 1—CBD Liposomal Formulations

### Liposome Preparation and Characterization

### Materials

Hydroxypropyl-β-cyclodextrin (HPCD) was obtained from roquette

Hydrogenated soy phosphatidylcholine (HSPC), and 1,2-dimyristoyl-sn-glycero-3-phosphocholine (DMPC) were obtained from Lipoid GmbH (Ludwigshafen, Germany)

Cannabidiol (CBD)—was obtained from THC Pharm (Batch. CBDAPI1802)

Ethanol absolute—was purchased from Merck

Solubilization enhancers: polyethylene glycol (PEG) 300, proplylene glycol (PG). Tween 80, and Dimethylacetamide (DMA) were purchased from Merck, Cremophor was obtained from Sigma.

### Methods:

### Liposome Preparation

Different types of phosphatidylcholines differing in their acyl-chains composition were used and tested. These include HSPC (mostly stearoyl, C18), DPPC dipalmitoyl, C16 DMPC (di-myristoyl, C14) and DOPC (Di-oleoyl, C18:1)) without cholesterol or with cholesterol (5 or 10%).

As the formulations with DOPC produced less favorable liposomes, these were excluded from further investigation.

The different liposomes prepared are detailed in Table 1.

Formulation F1 was prepared as follows: HSPC (S PC-3, Lipoid, Batch: 525600-2180662-01/042) and CBD (Batch: CBDAPI1802) were weighed in a vial. Ethanol absolute (Merck) was added and the vial and placed in a water bath at 65° C. until the solution was clear. Then, one ml of aqueous isotonic solution (for example dextrose 5%) was placed in the water bath. As soon as the lipid phase became clear, it was added to warmed water while stirring at 65° C. for 30 mm at 65° C.

Formulations containing CBD in the aqueous phase were prepared with the lipid phase similar to that prepared for F1. The aqueous phase was prepared by mixing all component of the specific aqueous phase followed by adding concentrated CBD solution in ethanol (700 mg/ml). The ethanolic solution is added slowly while stirring followed in some cases by short heating. As soon as the aqueous phase is clear or homogenously dispersed, the lipid phase is added slowly at 65° C. and stirring is continued for 30 min at 65° C.

### Release Assay

The release of CBD from the liposomes was determined at time point zero, 1 hour (in some cases) and 24 hours after incubation at 37° C. in the presence of 25% bovine serum in 25% sucrose. At each time point, total CBD and free CBD were determined as described below.

### Total CBD Assay

The liposomal CBD was diluted×20 in 25% serum and 25% sucrose which was further diluted in methanol and analyzed by HPLC under conditions known in the art for detection of CBD by HPLC.

### Free CBD Assay

The liposomal CBD was diluted×20 in 25% serum and 25% sucrose. This dilution was centrifuged, and the liposomes were floated on top of a clear phase. The lower clear phase (free CBD) was diluted in methanol and analyzed by HPLC.

### Lipid Concentration

Lipid concentration was determined by the modified Bartlett method and in some cases by HPLC method having evaporative light scattering (ELSD) detector.

### Results

### Formulations Containing Cholesterol in the Lipid Phase.

CBD formulations containing cholesterol in the lipid phase were prepared. CBD was solubilized in the lipid phase. Lipid phase in all tested formulations was 125 mg/ml (including CBD) and CBD was 70% molar of the lipid phase content. The formulations contained HSPC and DMPC with no cholesterol and with 5 and 10% molar cholesterol. These formulations contained CBD only in the lipid phase (not in the aqueous phase) and are described in Table 1.

The results show that HSPC exhibits a slightly slower release profile compared to DMPC liposomes. Cholesterol increased the release of CBD from the liposomes and this effect was more pronounced for DMPC.

Further formulations were therefore prepared with HSPC in the lipid phase (125 mg/ml lipid phase, 70% molar CBD) and different compositions of aqueous phase allowing solubilization or dispersion of CBD in the aqueous phase.

Table 2 shows two CBD formulations having CBD in aqueous phase containing only HPCD. The formulations resulted in similar loading (in terms of D/L ratio) and release profile as for F1 formulation (described in Table 1). This is probably a result of the law CBD concentrations that were able to be loaded to the aqueous phase containing only HPCD (8 mg/ml).

Table 3 describes CBD formulations having HPCD and surfactants (cremphor EL and Tween 80) in the aqueous phase allowing dispersion of 21 mg/ml CBD. These formulations did not result in higher D/L ratio and their release profile couldn't have been defined because they resulted in substantial amount of small liposomes that were not separated by our release method.

Table 4 presents formulations having HPCD and 25% PEG 300 in the aqueous phase allowing for 14 mg/ml CBD dispersion. Two of the formulations (A39 and A42) differed in their HPCD content resulted in substantially higher D/L ratios and slower release profile. Comparing these formulations with liposomes having the same aqueous composition without CBD in the aqueous phase resulted in much lower D/L ratio and faster release profile. Demonstrating that having substantial CBD fraction in the aqueous reservoir slowed down the release.

Table 5 describes CBD formulations having HPCD and 10-15% PG in the aqueous phase allowing for 21 mg/ml CBD dispersion. For each formulation a control formulation was also prepared having the same aqueous phase composition without CBD. The results showed that in all cases the release was slower in formulations having CBD in the aqueous phase and was more pronounced in cases the D/L ratio was substantially higher than control.

Without being bound thereto, it appears from the data presented herein that the addition of CBD to the aqueous phase that is enabled by the addition of co-solvents (the dispersing agent) or surfactants assist in delaying release of CBD from the liposome.

Further, without being bound thereto, it appears from the data presented herein that the addition of CBD to the lipid phase in the presence of cholesterol in an amount above 4% molar. e.g. 5-10% molar, results in rapid release of CBD from the liposomes (see Table 1, aqueous phase being DDW).

Further, without being bound thereto, it appears from the data presented herein that when CBD liposomes were prepared with CBD in the aqueous phase only, and the lipid phase was composed of lipids only, e.g. HSPC (without CBD), the release of CBD from the liposome was also rapid (78% free after 24 hours). Without being bound by theory, this may be as a result of lack of membrane stabilization when the lipid membrane does not contain CBD or some amount of cholesterol.

One conclusion that can arise from the findings disclosed herein that CBD in the lipid membrane stabilizes the membrane and thereby allows for the controlled release (prolonged) of CBD from the liposome.

TABLE 1
Liposomal-CBD containing cholesterol Composition and Characterization

| | Lipid | CBD | CBD | Lipid | | % Free CBD in release assay | | |
|---|---|---|---|---|---|---|---|---|
| No. | Phase | (mg/ml) | (mM) | (mM) | D/L | T = 0 | T = 1 h | T = 24 h |
| F1 | HSPC | 54.9 | 174.56 | >64 | >2.7 | 5.53 | ND | 25.6 |
| F16 | HSPC, 5% | 61.2 | 194.59 | 52.51 | 3.71 | 4.2 | 13.4 | 35.6 |
| | Chol. | | | | | | | |
| F17 | HSPC, 10% | 55 | 174.88 | 54.91 | 3.18 | 2.7 | 13.3 | 39.2 |
| | Chol. | | | | | | | |
| F18 | DMPC | 49 | 155.80 | 65.53 | 2.38 | 11.3 | 15 | 37.3 |
| F19 | DMPC, 5% | 46.9 | 149.13 | 60.19 | 2.48 | 12.2 | 25.2 | 84.7 |
| | Chol. | | | | | | | |
| F20 | DMPC, 10% | 47.5 | 151.03 | 46.15 | 3.27 | 7.14 | 34.8 | 74.5 |
| | Chol. | | | | | | | |

TABLE 2
Liposomal CBD formulations having CBD and in the lipid
phase (HSPC) and aqueous phases contain CBD and HPCD

| | | CBD | CBD | Lipid | | % Free | | |
|---|---|---|---|---|---|---|---|---|
| No. | Aq. Phase | (mg/ml) | (mM) | (mM) | D/L | t = 0 | t = 1 h | t = 24 h |
| F24 | 30% HPCD, | 30.02 | 95.47 | 37.20 | 2.57 | 0.55 | 6.41 | 21.4 |
| | 8 mg/ml CBD | | | | | | | |
| F25 | 5% HPCD, | 41.57 | 132.19 | 43.61 | 3.03 | 1.63 | 10.48 | 17.0 |
| | 8 mg/ml CBD | | | | | | | |

TABLE 3
Liposomal CBD formulations having CBD in the lipid phase (HSPC) and aqueous
phases contain CBD, HPCD and surfactants (cremophor or tween 80)

| | | CBD | CBD | Lipid | | % Free | | |
|---|---|---|---|---|---|---|---|---|
| No. | Aq. Phase | (mg/ml) | (mM) | (mM) | D/L | t = 0 | t = 1 h | t = 24 h |

| | | | | | | |
|---|---|---|---|---|---|---|
| F26 | 15% HPCD, 5% | 36.59 | 116.34 | 41.45 | 2.81 | Small liposomes |
| | Crem, 21 mg/ml | | | | | were also obtained, |
| | CBD | | | | | and the release |
| F27 | 2.5% HPCD. 5% | 60.28 | 191.66 | ND | ND | assay was not |
| | Crem, 21 mg/ml | | | | | suitable |
| | CBD | | | | | |
| F28 | 15% HPCD, 5% | 39.24 | 124.77 | 42.24 | 2.95 | |
| | Tw 80, 21 mg/ml | | | | | |
| | CBD | | | | | |
| F29 | 2.5% HPCD. 5% | 56.03 | 178.17 | 65.94 | 2.70 | |
| | Tw 80, 21 mg/ml | | | | | |
| | CBD | | | | | |

TABLE 4
Liposomal CBD formulations having CBD in the
lipid phase (HSBC) and aqueous phases contain
HPCD and PEG 300 with and without CBD.

| | | | | | | % Free | |
|---|---|---|---|---|---|---|---|
| | | CBD | CBD | Lipid | | t = | t = |
| No. | Aq. Phase | (mg/ml) | (mM) | (mM) | D/L | 0 | 24 h |
| A39 | 22.5% HPCD, | 43.9 | 139.59 | 37.73 | 3.70 | 3.84 | 9.59 |
| | 25% PEG 300 | | | | | | |
| | containing14 | | | | | | |
| | mg/ml CBD | | | | | | |
| A39 | 22.5% HPCD, | 26.6 | 84.58 | 44.33 | 1.91 | 3.42 | 16.2 |
| control | 25% PEG 300 | | | | | | |
| A40 | 11.25% HPCD, | 33.3 | 105.88 | 37.18 | 2.85 | 4.35 | 9.71 |
| | 25% PEG 300 | | | | | | |
| | containing14 | | | | | | |
| | mg/ml CBD | | | | | | |
| A41 | 3.75% HPCD, | 38.2 | 121.46 | 41.85 | 2.90 | 4.5 | 12.1 |
| | 25% PEG 300 | | | | | | |
| | containing14 | | | | | | |
| | mg/ml CBD | | | | | | |
| A42 | 1.9% HPCD, | 65.1 | 207.00 | 39.58 | 5.23 | 0.84 | 6.9 |
| | 25% PEG 300 | | | | | | |
| | containing14 | | | | | | |
| | mg/ml CBD | | | | | | |
| A42 | 1.9% HPCD, | 65.5 | 208.27 | 33.54 | 6.21 | 1.54 | 15.43 |
| control | 25% PEG 300 | | | | | | |

TABLE 5
Liposomal CBD formulations having CBD in the lipid phase (HSPC)
and aqueous phases contain HPCD and PG with and without CBD.

| | | Lipo- | | | | | |
|---|---|---|---|---|---|---|---|
| | | somal | | | | % Free | |
| | | CBD | CBD | Lipid | | t = | t = |
| No. | Aq. Phase | (mg/ml) | (mM) | (mM) | D/L | 0 | 24 h |
| A29 | 27% HPCD, | 110.8^{a} | 352.31 | 40.48 | 8.70 | 1.67 | 7.81 |
| | 10% PG, 21 | | | | | | |
| | mg/ml CBD | | | | | | |
| A29 | 27% HPCD, | 36.7 | 116.69 | 37.22 | 3.13 | 4.8 | 20.95 |
| control | 10% PG | | | | | | |
| A29.2 | 25.5% HPCD, | 64 | 203.50 | 40.94 | 4.97 | 3.14 | 13.26 |
| | 15% PG, 21 | | | | | | |
| | mg/ml CBD | | | | | | |
| A29.2 | 25.5% HPCD, | 35.7 | 113.51 | 41.25 | 2.75 | 5.31 | 21.51 |
| control | 15% PG | | | | | | |
| A29.3 | 27.8% HPCD, | 37.8 | 120.19 | 41.89 | 2.87 | 4.32 | 17.96 |
| | 7.5% PG, 21 | | | | | | |
| | mg/ml CBD | | | | | | |
| A29.3 | 27.8% HPCD, | 45.1 | 143.40 | 55.08 | 2.60 | 3.12 | 22.43 |
| control | 7.5% PG | | | | | | |
| A30 | 13.5% HPCD, | 41.1 | 130.68 | 37.86 | 3.45 | 3.56 | 17.26 |
| | 10% PG, 21 | | | | | | |
| | mg/ml CBD | | | | | | |
| A30 | 13.5% HPCD, | 36.6 | 116.38 | 37.86 | 3.07 | 3.91 | 27.35 |
| control | 10% PG | | | | | | |

^{a}The concentration is not reliable. Probably due to non-homogenous sampling

### Example 2—Liposomes Containing CBD-HSA

A liposomal formulation containing CBD-HSA as the aqueous phase was developed (Liposomal-CBD-HSA). For this formulation the aqueous phase was prepared by dispersing CBD in 5% has CBD was weighed in a vial and 5% HSA solution was added. The dispersion was stirred for at least two days at 4 C until homogeneous suspension was obtained with no particles observed on the walls of the vial. This dispersion was added to a warmed HSPC powder and stirred for 15 min at 65° C. An analytical method was developed that was able to differentiate between the liposomal bound and the HSA bound CBD. It was found that although the volume of the liposomes in the suspension is lower than the extra liposomal volume, most of the CBD is liposomal bound. Table 6 below shows the % liposomal CBD in different Liposomal-CBD-HSA formulations. The affinity of CBD to the liposomes and to HSA was therefore tested as detailed below.

Two formulation were prepared:
CBD-HSA preparation with empty MLV (40 mg/ml HSPC in 5% dextrose) at a 1:1 volumetric ratio
F1 formulation (CBD is only in the membrane lipids) with HSA solution at a 1:1 volumetric ratio.

The mixtures were placed in an incubator at 37° C. with 50 rpm shaking for 2 h. Table 7 presents the results. F1 formulation incubated with HSA showed only 1% of total CBD transferred to HSA from the liposomes. In the case of CBD-HSA incubated with empty MLV's, 35% of the CBD was transferred to the liposomes showing the much higher affinity of CBD to the lipids. These results were in line with the results obtained for the Liposomal-CBD-HSA formulations (Table 6) showing that CBD was mainly liposomal.

The addition of HSA to the liposomes allowed us to reach high D/L molar ratio in the liposomes.

TABLE 6
CBD distribution in different Liposomal-CBD-HSA formulations

| | | Total | | | % | |
|---|---|---|---|---|---|---|
| D/L | HSPC | CBD | Liposomal | | Liposomal | % CBD- |
| molar | conc. | conc. | CBD | CBD-HSA | CBD | HSA |
| ratio | (mg/g) | (mg/g) | (mg/ml) | (mg/ml) | (mg/ml) | (mg/ml) |
| 1.43 | 70.99 | 40.67 | 39.09 | 1.58 | 96.12 | 3.88 |
| 1.76 | 73.64 | 51.96 | 51.49 | 0.46 | 99.11 | 0.89 |
| 1.77 | 96.24 | 68.47 | 56.01 | 12.46 | 81.80 | 18.20 |
| 2.20 | 69.87 | 61.62 | 54.97 | 6.65 | 89.21 | 10.79 |
| 2.24 | 45.57 | 40.89 | 37.22 | 3.67 | 91.03 | 8.97 |
| 2.58 | 73.66 | 76.12 | 56.39 | 19.73 | 74.08 | 25.92 |
| 2.83 | 42.12 | 47.86 | 45.64 | 2.22 | 95.36 | 4.64 |
| 4.71 | 40.64 | 76.75 | 60.14 | 16.61 | 78.36 | 21.64 |
| 5.23 | 48.20 | 101.13 | 75.85 | 25.28 | 75.00 | 25.00 |

TABLE 7
Affinity of CBD to lipids vs HSA

| | Total | Lipo- | | | % | |
|---|---|---|---|---|---|---|
| | CBD in | somal | CBD- | Free | Lipo- | |
| | incubation | CBD | HSA | CBD | somal | % CBD- |
| | (mg/ml) | (mg/ml) | (mg/ml) | (mg/ml) | CBD | HSA |
| F1 | 15.02 | 14.75 | 0.15 | 0.12 | 98.22 | 1.01 |
| incubated | | | | | | |
| with HSA | | | | | | |
| CBD-HSA | 22.56 | 7.91 | 14.50 | 0.15 | 35.07 | 64.26 |
| incubated | | | | | | |
| with MLV's | | | | | | |

The release of CBD from the different formulations was tested in 50% adult bovine serum. In an HPLC vial, 50 mg formulation was weighed and 950 ul of 50:50 of serum: dextrose 5% solution was added. The mixture was vortexed and placed in an incubator at 37 C and 50 rpm shaking for 2 hr. The mixture was tested for total CBD content after dilution of 25-fold in methanol. The rest of the mixture was transferred to an Eppendorf and centrifuged (30 min, 14.000 rpm, 4° C.) and the upper phase was diluted 10-fold in methanol and HPLC analyzed. The in vitro release of several Liposomal-CBD-HSA formulations differing in their lipid and CBD content was tested and described in Table 8. The in vitro release test demonstrated slower release with increasing CBD concentrations in the formulation.

TABLE 8
Release of CBD in 50% serum

| | % Release from | | % Release from |
|---|---|---|---|
| Total CBD | liposomes | Total CBD | liposomes |
| conc. | containing HSPC | conc. | containing HSPC |
| (mg/ml) | 41-48 mg/ml | (mg/ml) | 70-74 mg/ml |
| 38.3 | 76.5 | 40.7 | 71.0 |
| 46.4 | 70.4 | 52.0 | 70.8 |
| 59.5 | 72.2 | 61.6 | 66.4 |
| 68.4 | 59.1 | 76.1 | 60.6 |
| 86.9 | 56.4 | 97.8 | 59.0 |

### Example 3—In Vivo PK Studies of CBD Formulations

Two in vivo studies were performed to study the plasma profile and residual CBD in muscles after IM injection of different CBD formulations: the 1^{st} study tested 4 formulations up to 3 days and the second study tested 4 formulations for up to 3 weeks. Detailed description of each study is found below.

### 1^{st} Study

### Formulations Preparation and Characterization

Details regarding the materials used for the formulation's preparations are summarized in Table 9. All formulations were prepared under aseptic conditions in a biological hood using autoclaved equipment to ensure aseptic preparations.
a. Free CBD in PG: CBD was prepared at a concentration of 50 mg/g PG and vortexed until a clear solution was obtained.
b. F1: Liposomal formulation of CBD in which CBD is solubilized only in membrane phospholipid of the liposomes: CBD and HSPC were solubilized together in ethanol at 65° C. (lipid phase) until a clear solution was obtained. The lipid phase was added to a 5% dextrose solution at 65° C. while stirring and left for stirring for 30 min (at 65° C.). The obtained multi-lamellar liposomes (MLV) formulation was then washed with 5% dextrose solution until the osmolality of the preparation was iso-osmotic.
c. F-HPCD-PEG: Liposomal formulation of CBD in which CBD is solubilized in both liposome membrane phospholipid and it was also dispersed in the intra-liposomal aqueous phase using the solubilizing agents: HPCD and PEG 300. CBD and HSPC were solubilized in ethanol at 65° C. (lipid phase) until a clear solution was obtained. Aqueous phase was prepared by adding CBD solution in ethanol into a solution containing 27% (w/w) HPCD and 10% (w/w) PEG 300 at 65° C. The aqueous phase was almost clear. The lipid phase was added to the aqueous phase at 65° C. while stirring and left for stirring for 30 min (at 65° C.). The obtained formulation was then washed with 5% dextrose solution until the osmolality of the preparation was iso-osmotic.
d. Liposomal-CBD-HSA: CBD was firstly dispersed in 5% HSA solution. This dispersion was added to a warmed HSPC and stirred for 15 min at 65° C.

TABLE 9
materials

| Material | Details |
|---|---|
| CBD | THC Pharma, Batch CBDAPI1805 |
| Hydrogenated Soy | Lipoid, 525600-2180662-01/042 |
| Phosphatidylcholine | |
| (HSPC) | |
| Ethanol absolute | Biolab, lot 1287341 |
| Hydroxypropyl beta | Kleptose, HPB parenteral, batch E0252 |
| cyclodextrin (HPCD) | |
| Polyethylene glycol (PEG) | Merck Emprove, 8.17002.5000, K50758402 |
| 300 | 848 |
| Human Serum Albumin | Alburex 50 g/l, lot P100020267 |
| (HSA) | |
| Propylene Glycol (PG) | Merck, 1.07478.2500, K47269178 547 |

### Formulations Characterization

### Assay for CBD

Total and free CBD content was determined by HPLC method. The chromatographic conditions used were based on USP method for Dronabinol and summarized in Table 10.

Sample preparation for the analysis varied for each formulation and described below.
Total CBD concentration was similar for all formulations. Specifically, 10-20 mg of formulation was weighed into a 10 ml volumetric flask. Methanol was added to line. After vortex, sample was centrifuged, and the upper phase was analyzed.
Free CBD content was tested for F1, and Liposomal CBD-HSA: A 200 μl formulation was placed in an Eppendorf and centrifuged for 30 min at 40 C, 14,000 rpm. The clear upper phase was then diluted 10-fold with methanol followed by vortex and centrifugation (14,000 rpm, 10 min, 40 C). Upper phase was HPLC analyzed.
Albumin bound CBD was quantified for Liposomal CBD-HSA formulations: This method was developed to enable separation between liposomal and albumin-bound CBD. For this purpose, isosmotic medium, allowing density-based separation was used. Medium was prepared with 1.5 g dextrose (Sigma, D9434, batch 119K0042) and 10 g Ficoll 400 (Sigma, F-4375, lot 29C-0095) solubilized in 50 ml DDW (volumetric flask). Medium osmolality was 290 mOsm/kg. 50 mg formulation was placed in an Eppendorf tube and 1.5 ml medium was added. The tube was vortexed and then centrifuged (4° C., 30 min, 14,000 rpm). The upper phase of the tube was cut, and all liquid remained in the lower part containing the precipitate was removed. The precipitation was transferred to another Eppendorf and 1 ml methanol was added. After vortex and centrifugation, the upper phase was diluted 10-fold with methanol.
The IV formulation in Cremophore: ethanol was tested for total content as described above for Total CBD concentration. The appearance after dilution with saline was examined to follow formulation behavior for injection and ensure no precipitation. The formulation was diluted×10 with saline and after 1 h (the time allowed for the formulation to be injected after preparation), the appearance was recorded.

TABLE 10
Chromatographic conditions of CBD assay

| | |
|---|---|
| Mobile phase | Methanol:DDW:tetrahydrofuran:acetonitrile |
| | at a volumetric ratio of 45:25:20:10 |
| Column | Phenomemex C18, 150 × 4.6 mm |
| Detector | UV 228 nm |
| Flow rate | 1 ml/min |
| Injection vol. | 20 μl |
| Column temp | 25° C. |

### Release Assay

The release of CBD from the different formulations was tested in 50% adult bovine serum. In an HPLC vial, 50 mg formulation was weighed and 950 ul of 50:50 of serum: dextrose 5% solution was added. The mixture was vortexed and placed in an incubator at 37 C and 50 rpm shaking for 2 hr. The mixture was tested for total CBD content after dilution of 25-fold in methanol. The rest of the mixture was transferred to an Eppendorf and centrifuged (30 min, 14,000 rpm, 4° C.) and the upper phase was diluted 10-fold in methanol and HPLC analyzed.

### Particle Size Measurement

Particle size was determined using Coulter LS 130.

### Osmolality

Osmolality was measured by freeze point method using Advanced instrument, Model 3320 osmometer.

### Lipid Concentration

Lipid concentration was determined by HPLC/ELSD method.

### Microscopical Observation

The formulations were observed under optical microscope (Zeiss SN 221209). Few fields were observed, and a representative picture was taken for each formulation.

### Injectability

One ml syringe was filled with 0.3-0.5 ml of formulation. A 25G needle was connected to the syringe and the injected volume of the formulation without being stuck was determined. The process was repeated for three times.

### Sterility

One vial from each formulation was tested by the microbiology unit in Hadassah. Aliquot from each vial was plated on blood agar and chocolate agar and placed at room temperature and at 37° C. incubator.

### In Vivo Study Protocol

A total of 36 female BALB/C mice aged 12 weeks were injected IM with a single dose of each formulation (9 mice per group) at injection volume of ˜50 μl (˜2.5 ml/kg at 1 injection site) for all formulations. F1 having lower CBD concentration, was injected at 2 injection sites at a total injection volume of ˜100 μl. In order to ensure the exact dose administered, the syringe was weighed before and after injection and the actual amount injected was recorded.

At the time-points detailed below, 3 mice of each group were euthanized with CO₂ and terminal blood was immediately collected from the retro-orbital sinus in labeled 0.5 ml K3EDTA blood collection tubes (Mini Collect, Greiner-bio-one, Austria). The blood was centrifuged at 2000 g for 10 minutes before plasma was extracted, collected in labeled tubes and frozen at −20° C. immediately after collection. The samples were then stored at −80° C. pending analysis.

After blood collection, the quadriceps femoris muscles were collected into pre-weighed 15 ml tubes.

Time-points for the blood and muscles collection were: 2 hours, 24 hours and 72 hours after injection.

### Bioanalytical Assays

### Assay for CBD in Plasma

CBD was extracted from plasma samples that were spiked with cannabigerol (CBG, 1 mg/ml in methanol, Sigma, Cat. C-141-1) used as internal standard (IS) followed by dilution of the plasma five-fold in acetonitrile. After vigorous vortex it was centrifuged, and the upper phase was analyzed. Final IS concentration in the samples was 100 ng/ml.

Plasma extracts were analyzed by LCMS method. Specifically, LC-MS/MS analyses were conducted on a Sciex (Framingham, Mass., USA) Triple Quad™ 5500 mass spectrometer coupled with a Shimadzu (Kyoto, Japan) UHPLC System. The concentrations were calculated based on a calibration curve of CBD in plasma at a range of 1-1,000 ng/ml having 100 ng/ml of IS.

CBD spiking solutions for the preparation of calibration curve in plasma were prepared in acetonitrile. CBG was prepared in methanol.

### Assay for CBD in Muscles (Site of Injection)

Muscles were removed surgically, and their weight was recorded. Thereafter, 2 ml of 15% collagenase solution (Sigma, C7657) were added and tubes were incubated overnight at 37° C. After incubation, 8 ml of acetonitrile were added, vortexed and centrifuged. Upper phase was HPLC analyzed. Chromatographic conditions were described in Table 10.

The concentration of CBD in each muscle was calculated based on a calibration curve of CBD in acetonitrile.

The recovery of CBD from muscles was determined for each formulation following spiking of CBD formulations into muscles compared to spiking into acetonitrile.

### Results

The formulations were characterized for total CBD and HSPC content and their molar ratio, particle size and appearance in the microscope. CBD concentration was in

TABLE 11
Formulations characterization

| | | | Muscle conc. | Plasma conc. |
|---|---|---|---|---|
| Formulation | Time point | | (μg/g) | (ng/ml) |
| Free CBD in PG | 2 | h | 6,152 | 506 |
| | | | 3,905 | 707 |
| | | | 3,144 | 1,344 |
| | 24 | h | 3,686 | 140 |
| | | | 2,747 | 365 |
| | | | 3,901 | 208 |
| | 72 | h | 3,148 | 76.6 |
| | | | 3,449 | 65.7 |
| | | | 2,588 | 124 |
| F1 | 2 | h | 2,750 | 2,269 |
| | | | 3,433 | 2,688 |
| | | | 2,335 | 2,117 |
| | 24 | h | 1,480 | 236 |
| | | | 2,018 | 179 |
| | | | 1,025 | 249 |
| | 72 | h | 2,704 | 56.1 |
| | | | 1,841 | 84.6 |
| | | | 2,231 | 90.7 |
| F-HPCD-PEG | 2 | h | 2,015 | 571 |
| | | | 455 | 149 |
| | | | 966 | 410 |
| | 24 | h | 1,225 | 46.3 |
| | | | 2,035 | 58.4 |
| | | | 2,670 | 115 |
| | 72 | h | 3,732 | 34.1 |
| | | | 5,452 | 49.2 |
| | | | 5,435 | 13.3 |
| Liposomal-CBD-HSA | 2 | h | NC | 663 |
| | | | 4,979 | 554 |
| | | | 5,310 | 889 |
| | 24 | h | 5,187 | 90.8 |
| | | | 5,310 | 77.6 |
| | | | 3,643 | 59.3 |
| | 72 | h | 2,504 | 23.4 |
| | | | 6,674 | 35.3 |
| | | | 4,411 | 88 |

the range of 50-60 mg/g for all formulations with the exception of F1 having 30 mg/g CBD content. These results are summarized in Table 11.

Mice were injected IM with the prepared formulations. The syringe weight was recorded before and after injection to accurately calculate the amount of injected dose.

Table 12 summarizes the plasma and muscle concentrations obtained. This study showed clearly that CBD retained at the site of injection (muscle) for more than 72 hours. And during all this time the depot in the muscle is releasing CBD to the plasma at a rate dependent of the formulation used.

TABLE 12
Plasma and muscle concentrations obtained

| | | HSPC | Drug to | | |
|---|---|---|---|---|---|
| | CBD | concen- | lipid | | Mean |
| | content | tration | molar | Osmolality | size |
| Formulation | (mg/g) | (mg/ml) | ratio | (mOsm/kg) | (μm) |
| Free CBD in PG | 50.16 | ND | ND | ND | ND |
| F1 | 29.52 | 39.82 | 1.85 | 336 | 11.03 |
| F-HPCD-PEG | 50.35 | 91.68 | 1.89 | 302 | 5.99 |
| Liposomal-CBD- | 60.70 | 92.13 | 1.65 | 277 | 8.93 |
| HSA | | | | | |

NC = not calculated

### 2^{nd} Study

### Formulations Preparation and Characterization

### Materials and Methods

### Materials

Details regarding the materials used for the formulation's preparations are summarized in Table 13.

TABLE 13
Material details

| Material | Details |
|---|---|
| CBD | THC Pharma, Batch CBDAPI1805 |
| Hydrogenated Soy | Lipoid, 525600-2180662-01/042 |
| Phosphatidylcholine (HSPC) | |
| Ethanol absolute | Biolab, lot 1287341 |
| Human Serum Albumin (HSA) | Alburex 50 g/l, lot P100020267 |
| Cremophor EL | Sigma, C5135, lot BCBX1177 |
| Propylene Glycol (PG) | Merck, 1.07478.2500, K47269178 547 |

### Methods

### Formulations Preparation

All formulations were prepared under aseptic conditions in a biological hood using autoclaved equipment to ensure aseptic preparations. Three formulation types were used for the Intramuscular (IM) pharmacokinetic (PK) study.

CTRL-PG: Control formulation of CBD solubilized in propylene-glycol (PG). CBD was prepared at a concentration of 50 mg/g PG and vortexed until a clear solution was obtained.

F1: Liposomal formulation of CBD in which CBD is solubilized only in membrane phospholipid of the liposomes. CBD and HSPC were solubilized together in ethanol at 65° C. (lipid phase) until a clear solution was obtained. The lipid phase was added to a 5% dextrose solution at 65° C. while stirring and left for stirring for 30 min (at 65° C.). The obtained multi-lamellar liposomes (MLV) formulation was then washed with 5% dextrose solution until the osmolality of the preparation was iso-osmotic.

Liposomal-CBD-HSA: Liposomal formulations of CBD in which CBD was dispersed firstly in HSA followed by passively encapsulated the CBD-HSA in liposomes. CBD was firstly dispersed in 5% HSA solution. This dispersion was added to a warmed HSPC and stirred for 15 min at 65° C.

IV formulation: The formulation used for IV administration was 10 mg/g CBD formulation solubilized in Cremophor:Ethanol 50:50 solution. This formulation was diluted 10-fold with saline prior to injection to result in 1 mg/ml post dilution concentration. The diluted formulation was used within 1 h after preparation.

In the following, the formulations are defined by the CBD amount/ml solution containing always 50 mg protein. Thus, for example, “liposomal CBD/HSA 50 mg/ml” denotes a liposomal formulation comprising 50 mg CBD and 50 mg HSA.

### Formulations Characterization—as Described for the 1^{st} Study

### In Vivo Study Protocol

### IV Administration

A total of 18 female BALB/C mice aged 12 weeks were injected IV with a single dose of 10 mg/kg CBD formulation in cremophor:ethanol.

At the time-points detailed below, 3 mice were euthanized with CO₂ and terminal blood was immediately collected from the retro-orbital sinus in labeled 0.5 ml K₃EDTA blood collection tubes (Mini Collect, Greiner-bio-one, Austria). The blood was centrifuged at 2000×g for 10 minutes before plasma was extracted, collected in labeled tubes and frozen at −20° C. immediately after collection. The samples were then stored at −80° C. pending analysis.

Time-points for the blood collection: 2 min, 1 hour, 4 hours, 8 hours, 24 hours and 48 hours.

### IM Administration

A total of 36 female BALB/C mice aged 12 weeks were injected IM with a single dose of the IM formulations. Nine mice per formulation. The syringe was weighed before and after injection to enable precise recording of the exact volume and hence dose that each mouse received. Details regarding the injection volumes and estimated doses for each group are summarized in Table 14.

Two mice were not injected with formulations and were used as control for body weight (BW) change over time.

At the time-points detailed below, 3 mice of each group were euthanized with CO₂ and terminal blood was immediately collected from the retro-orbital sinus in labeled 0.5 ml K₃EDTA blood collection tubes (Mini Collect, Greiner-bio-one, Austria). The blood was centrifuged at 2000×g for 10 minutes before plasma was extracted, collected in labeled tubes and frozen at −20° C. immediately after collection. The samples were then stored at −80° C. pending analysis.

After blood collection, the quadriceps femoris muscles were collected into pre-weighed 15 ml tubes.

Time-points for the blood collection: 72 hours, 1 week and 3 weeks after injection.

Mice weight was recorded before dosing and before euthanization. Mice sacrificed at 3-week time point were also weighed two weeks after administration.

TABLE 14
Injection volumes and estimated doses for each group

| | Injection | No. of | Estimated dose |
|---|---|---|---|
| | volume per | injected | (mg/kg) assuming |
| Group | muscle (ml) | muscles | 20 g mouse |
| Free CBD in PG | 0.05 | 1 | 131 |
| F1 | 0.05 | 2 | 107 |
| Lipo-CBD-HSA 50 mg/ml | 0.05 | 1 | 125 |
| Lipo-CBD-HSA 100 mg/ml | 0.05 | 2 | 490 |

### Results

The present Example defined pharmacokinetic profile of three particle-based CBD formulations vs. a solution of CBD in propylene glycol (PG) after IM administration or IV administration (administration dose of 12 mg/kg, that based on the literature was effective in several animal models 1-3).

### Formulations

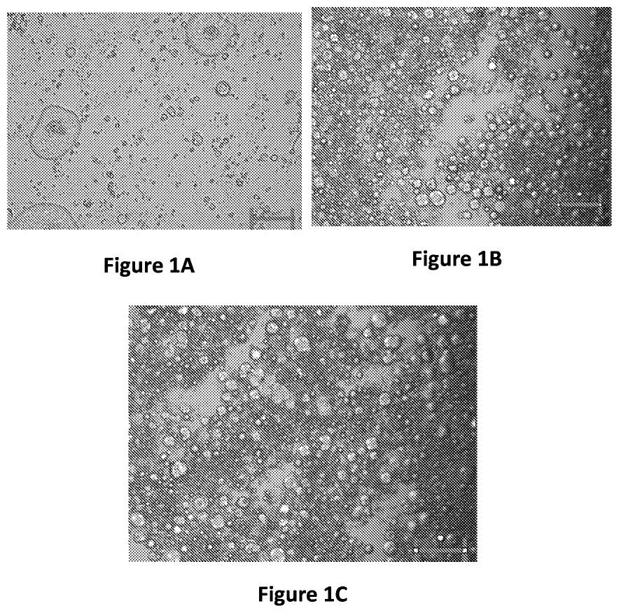

Liposomal CBD-HSA formulations were prepared by hydrating HSPC with CBD-HSA dispersion at 65° C. The obtained liposomes were spherical and homogenous as can be observed by the microscope image (FIG. 1B-1C).

The mean diameter of the liposomes was 8.1 μm for the 50 mg/ml formulation and 6.7 μm for the 100 mg/ml formulation.

CBD concentrations in the formulations was the expected concentrations (based on calculations). The 100 mg/ml formulation presented a high molar drug to lipid (D/L) ratio of 3.05. CBD in these formulations appeared to be distributed between the liposomal CBD (both in the membrane and in the internal aqueous phase) and with the albumin (CBD-HSA) outside the liposomes.

The formulation characterization is provided in Table 15 and the particle size is summarized in Table 16.

TABLE 15
Formulations characterization (IM administration)

| | Total | Free | | D/L | | |
|---|---|---|---|---|---|---|
| | CBD | CBD | HSPC | molar | % | Inject- |
| | (mg/g) | (mg/g) | (mg/ml) | ratio | Bound* | ability |
| Free CBD | 52.3 | NA | NA | NA | NA | NA |
| in PG | | | | | | |
| F1 | 21.3 | 0.02 | ND | ND | 51 | Freely** |
| Lipo-CBD- | 50.16 | 0.310 | 82.87 | 1.51 | 30 | Freely |
| HSA | | | | | | |
| 50 mg/ml | | | | | | |
| Lipo-CBD- | 97.98 | 0.324 | 80.03 | 3.05 | 43 | Freely |
| HSA | | | | | | |
| 100 mg/ml | | | | | | |

*Bound at after 2 hr, in 50% serum
**freely means that all the volume of the formulation was immediately/freely injected from the syringe

It is noted that all formulations were also found to be sterile, i.e. no microbial growth was detected in any of the tested formulations.

The CBD formulation in Cremophor:ethanol for IV administration was also characterized. The concentration of CBD in the concentrate was 11.7 (mg/ml). After dilution with saline, the solution was clear for at least 1 hour.

TABLE 16
Particle size (mean, d10, d50 and d90)

| | Mean | Span* | <10% | <50% | <90% |
|---|---|---|---|---|---|
| F1 | 9.2 | 2.5 | 1.9 | 7.1 | 19.8 |
| Lipo-CBD-HSA 50 mg/ml | 8.1 | 2.7 | 1.6 | 6.1 | 18.2 |
| Lipo-CBD-HSA 100 mg/ml | 6.7 | 2.9 | 1.3 | 4.9 | 15.5 |

*Span = (D90-D10)/D50

The distribution of CBD between the liposomes (both membranal and intra-liposomal core) and the CBD-HSA in these formulations was determined and is summarized in Table 17.

TABLE 17
CUD distribution in Lipnsomal-CBD-HSA formulations

| | | % Liposomal | % Albumin |
|---|---|---|---|
| | % Free CBD | CBD | bound |
| Lipo-CBD-HSA | 0.62 | 85.62 | 13.76 |
| 50 mg/ml | | | |
| Lipo-CBD-HSA | 0.33 | 90.72 | 8.95 |
| 100 mg/ml | | | |

Table 17 shows that although the liposomal volume was lower than the extra-liposomal volume, most of the CBD was liposomal (86-91%) and relatively a small fraction was bound to HSA outside the liposomes (9-14%). It was therefore assumed that most of the HSA-bound CBD was transferred into the liposome-forming lipids. This observation was in accordance with the partitioning of CBD between HSA and lipids as described in Table 6 of Example 2.

Following 2 hr of incubation in the presence of 50% serum, the free CBD concentrations were 33 and 53 mg/ml for 50 mg/ml and 100 mg/ml preparations accounting for 70 and 57% release (100-% bound=% release). This release rate was closer to the rate obtained for F1 liposomes. F1 was a liposomal formulation in which the CBD was solubilized with the membrane lipids and is probably located only in the liposomal membrane. CBD concentration in this formulation was 21.3 mg/ml, lower than other formulations, due to the wash steps required for the removal of the ethanol from the formulation. F1 appearance under the microscope demonstrated small round particles, relatively in distance from each other (FIG. 1A). Mean diameter was 9.2 μm (Table 16). The release in serum was 49%, similar to that of liposomal-HSA formulations (Table 15).

The reference IM formulation used was a solution of CBD in propylene glycol. An additional reference included a group of mice dosed IV with 12 mg/g dose (Cremophore:ethanol formulation diluted with saline prior to injection).

### PK Profile

The PK profile obtained after IV administration of 12 mg/kg CBD dose is summarized in Table 18.

TABLE 18
Plasma CBD profile after IV administration of 12 mg/kg dose

| | | | Mean plasma CBD |
|---|---|---|---|
| | | | concentration normalized |
| Time point | | Mean ± SD (ng/ml) | to dose (±SD)(ng/ml/mg/kg) |
| 5 | min | 8,856 ± 1,451 | 757 ± 124 |
| 1 | h | 177 ± 40 | 15 ± 3.46 |
| 4 | h | 35 ± 11 | 2.99 ± 0.90 |
| 8 | h | 9.50 ± 3.67 | 0.81 ± 0.31 |
| 24 | h | BLOD | BLOD |
| 48 | h | BLOD | BLOD |

*BLOD—below limit of detection

Table 18 shows that CBD concentrations decreased rapidly from 8,856 ng/ml at 5 min to 9.5 ng/ml, 8 hr after administration. At the late time points (24 hr and 48 hr) CBD concentrations were below the limit of detection (BLOD).

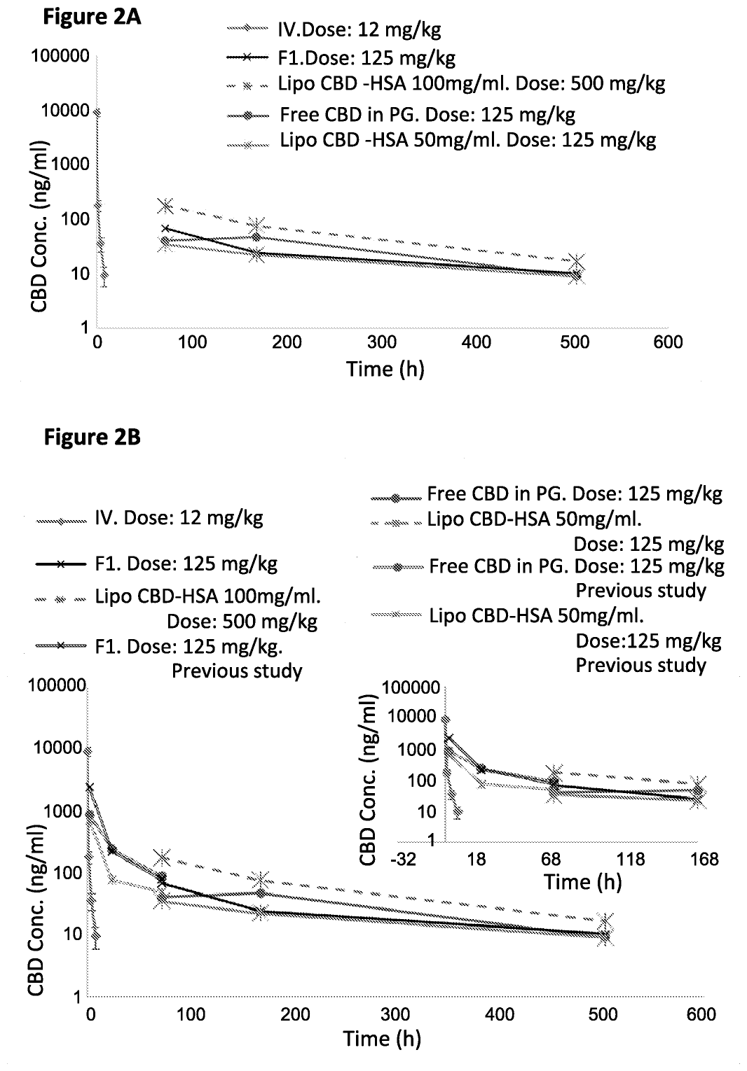

Plasma concentrations obtained after IM administration are summarized in Tables 19A-19B and FIG. 2.

TABLE 19A
CBD plasma concentrations across IM formulations

| | | | | | Mean (±SD) |
|---|---|---|---|---|---|
| | | | | Plasma conc. | plasma conc. |
| | | | | Normalized | Normalized |
| | Time | CBD | Plasma | to dose | to dose |
| Formu- | point | dose | conc. | (ng/ml/ | (ng/ml/ |
| lation | (days) | (mg/kg) | (ng/ml) | mg/kg)* | mg/kg)** |
| Free CBD | 3 | 156 | 34 | 0.218 | 0.254 ± 0.039 |
| in PG | | 154 | 38 | 0.249 | |
| | | 160 | 47 | 0.295 | |
| | 7 | 131 | 29 | 0.220 | 0.326 ± 0.110 |
| | | 130 | 42 | 0.319 | |
| | | 160 | 70 | 0.439 | |
| | 21 | 138 | 9.2 | 0.067 | 0.063 ± 0.005 |
| | | 127 | 0 ^{#} | 0.000 ^{#} | |
| | | 148 | 8.9 | 0.060 | |
| F1 | 3 | 113 | 81 | 0.717 | 0.612 ± 0.114 |
| | | 108 | 68 | 0.628 | |
| | | 113 | 55 | 0.490 | |
| | 7 | 115 | 25 | 0.222 | 0.201 ± 0.039 |
| | | 121 | 19 | 0.157 | |
| | | 121 | 27 | 0.226 | |
| | 21 | 115 | 12 | 0.101 | W/o outlier |
| | | 109 | 83^{##} | 0.760^{##} | 0.089 ± 0.017 |
| | | 115 | 8.8 | 0.077 | 0.313 ± 0.388 |

*Plasma concentration normalized to dose (ng/ml/mg/kg)
**Mean ± SD plasma concentration normalized to dose (ng/ml/mg/kg)
^{#} Presumed to be error with injection (thus not included in mean calculation)
^{##}Outlier. Defined as having value two-fold higher than the average value of the group.

TABLE 19B
CBD plasma concentrations across IM formulations

| | | | | | Mean (±SD) |
|---|---|---|---|---|---|
| | | | | Plasma conc. | plasma conc. |
| | | | | Normalized | Normalized |
| | Time | CBD | Plasma | to dose | to dose |
| Formu- | point | dose | conc. | ng/ml/ | (ng/ml/ |
| lation | (days) | (mg/kg) | (ng/ml) | mg/kg)* | mg/kg)** |
| Lipo- | 3 | 150 | 26 | 0.175 | 0.248 ± 0.074 |
| CBD-HSA | | 118 | 38 | 0.324 | |
| 50 mg/ml | | 157 | 38 | 0.245 | |
| | 7 | 133 | 15 | 0.116 | 0.166 ± 0.047 |
| | | 136 | 29 | 0.210 | |
| | | 129 | 22 | 0.171 | |
| | 21 | 137 | 11 | 0.077 | 0.063 ± 0.014 |
| | | 120 | 7.6 | 0.063 | |
| | | 181 | 8.7 | 0.048 | |
| Lipo- | 3 | 500 | 109 | 0.218 | 0.316 ± 0.086 |
| CBD-HSA | | 614 | 217 | 0.354 | |
| 100 mg/ml | | 521 | 196 | 0.376 | |
| | 7 | 567 | 86 | 0.151 | 0.138 ± 0.022 |
| | | 513 | 58 | 0.112 | |
| | | 550 | 83 | 0.150 | |
| | 21 | 463 | 13 | 0.029 | 0.033 ± 0.006 |
| | | 541 | 22 | 0.040 | |
| | | 513 | 15 | 0.030 | |

*Plasma concentration normalized to dose (ng/ml/mg/kg)
**Mean ± SD plasma concentration normalized to dose (ng/ml/mg/kg)

Tables 19A-19B and FIG. 2A show that the plasma concentrations after administration of all IM formulations and up to 3 weeks after administration were in the range of the IV profile obtained 1-8 hr after administration. This means, prolonged delivery by the IM injection of the formulations. FIG. 2B contains also the PK data from the 1^{st} study (referred to as “previous study” in the Figure) for the same formulations tested in the 2^{nd} study, however, from time point T=2 hours and 24 hours, therefore, providing us full overview of the CBD plasma profile after IV or IM administrations.

Interestingly, while after V administration of IV therapeutic dose of CBD high levels of CBD plasma are obtained immediately after administration, the plasma level decrease within several hours; yet, with IM administration of high CBD dose, initial levels of plasma CBD are similar to that of IV dose (e.g. after 2 hours), the levels are then essentially maintained high within a period of at least 3 weeks, for all IM formulations.

Moreover, the decrease in plasma levels was very slow with less than one order of magnitude decrease obtained over 3 weeks, for all formulations. This slow decrease, compared to the rapid decrease of the IV formulation, demonstrates that the terminal slope of the IM profile is not elimination dependent but rather absorption dependent showing that the formulations are continuously releasing CBD from the muscles over this long period.

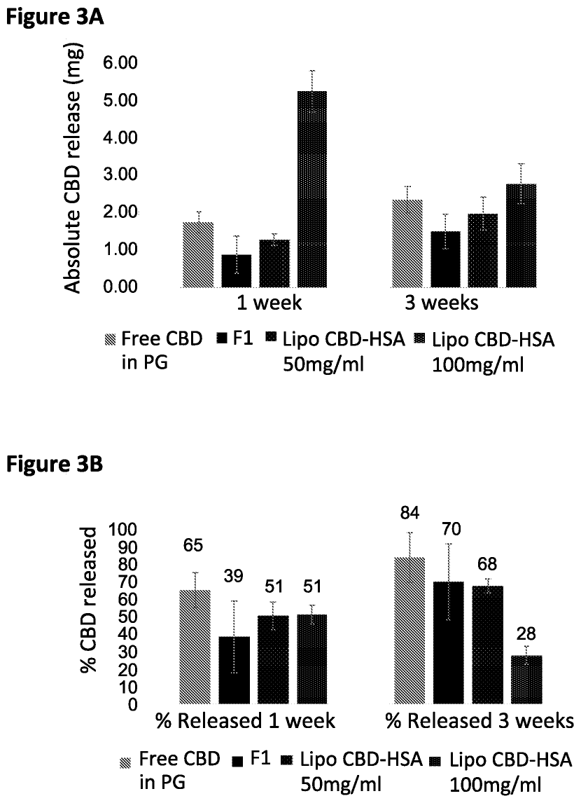

Table 20 summarize the residual content of CBD in the muscles compared to the initial CBD administered to each mouse, and FIGS. 3A-3B presents the average CBD released from the muscles per group compared to the initial administered CBD.

TABLE 20
CBD muscle concentrations (N = 3 per group)

| | | | | | CBD | | |
|---|---|---|---|---|---|---|---|
| | | | | | released | | Plasma CBD |
| | | Absolute | Absolute | % CBD | from | Estimated | normalized to |
| | | CBD | CBD in | released | muscle | daily CBD | estimated CBD |
| | Time | administered | muscle | from | per day | dose | daily dose |
| Formulation | (days) | (mg) | (mg) | muscle ^{a} | (mg/day) | (mg/kg)^{b} | (ng/ml/mg/kg) |
| Free CBD | 7 | 2.60 | 0.59 | 77 | 0.29 | 14.5 | 2.00 |
| in PG | | 2.44 | 0.98 | 60 | 0.21 | 10.5 | 4.00 |
| | | 3.06 | 1.26 | 59 | 0.26 | 13.0 | 5.38 |
| | 21 | 2.79 | 0.18 | 94 | 0.12 | 6.0 | 1.53 |
| | | 2.54 | 0.03^{#} | 99^{#} | 0.12^{#} | 6.0 | NC |
| | | 2.86 | 0.74 | 74 | 0.10 | 5.0 | 1.78 |
| F1 | 7 | 2.27 | 1.47 | 35 | 0.11 | 5.5 | 4.55 |
| | | 2.21 | 1.77 | 20 | 0.06 | 3.0 | 6.33 |
| | | 2.35 | 0.93 | 61 | 0.20 | 10.0 | 2.70 |
| | 21 | 2.18 | 0.34 | 85 | 0.09 | 4.5 | 2.67 |
| | | 2.16 | 0.44 | 80 | 0.08 | 4.0 | NC |
| | | 2.20 | 1.21 | 45 | 0.05 | 2.5 | 3.52 |
| Lipo-CBD- | 7 | 2.66 | 1.43 | 46 | 0.18 | 9.0 | 1.67 |
| HSA | | 2.55 | 1.38 | 46 | 0.17 | 8.5 | 3.41 |
| 50 mg/ml | | 2.43 | 0.97 | 60 | 0.21 | 10.5 | 2.10 |
| | 21 | 2.86 | 1.06 | 63 | 0.09 | 4.5 | 2.44 |
| | | 2.38 | 0.71 | 70 | 0.08 | 4.0 | 1.90 |
| | | 3.55 | 1.06 | 70 | 0.12 | 6.0 | 1.45 |
| Lipo-CBD- | 7 | 10.46 | 4.72 | 55 | 0.82 | 41.0 | 2.10 |
| HSA | | 10.27 | 5.61 | 45 | 0.67 | 33.5 | 1.73 |
| 100 mg/ml | | 9.98 | 4.62 | 54 | 0.77 | 38.5 | 2.16 |
| | 21 | 9.29 | 6.37 | 31 | 0.14 | 7.0 | 1.86 |
| | | 10.53 | 7.30 | 31 | 0.15 | 7.5 | 2.93 |
| | | 9.80 | 7.61 | 22 | 0.10 | 5.0 | 3.00 |

${}^{a} \% CBD released from muscle = \frac{CBD administered - CBD in muscle}{CBD administered} \times 100$ ^{b}Estimated daily CBD dose (mg/kg) calculated for 20 g mouse = CBD released per day/0.02 kg
* An outlier. Not included in the calculation of the mean
^{#}Probably a problem with the injection. Not included in the calculation of the mean
NC Not calculated due to outlier value of muscle and plasma concentration

At 1-week time point differences were found between groups showing more release for the high dose groups. These differences were not found for the 3-weeks' time point. The amount of CBD released from the muscles was normalized to the days from administration to estimate the amount CBD released per day and hence the dose of CBD that reached the circulation per day (assuming 20 g mouse). When plasma levels were normalized to this estimated daily dose, the mean normalized values were similar across the groups and were in the range of 1.7-4.5 ng/ml/mg/kg. These values are similar to the IV values obtained following 4-8 h after administration (Table 18) and demonstrate that the plasma concentrations are dependent on CBD released from the muscles and may be thus control by the formulations. From the % CBD release data, CBD reservoir in the muscle may be calculated for each formulation. free CBD and the liposomal formulations released the majority of the CBD at the 3 weeks time point (not including the unexplained low value for liposomal CBD-HSA 100 mg/ml, 3 weeks).

TABLE 21
Pharmacokinetic parameters obtained following
CBD IV administration of 12 mg/kg dose

| Parameter | result |
|---|---|
| T_{1/2} (hr) | 1.68 |
| Tₘₐₓ (hr) | 0.083 |
| Cₘₐₓ (ng/mL) | 8,856 |
| Cₘₐₓ/Dose (ng/mL)/(mg/kg) | 738 |
| C0 (ng/mL) | 9,464 |
| AUC₀_{_{—}}_{Tlast} (hr*ng/mL) | 5,000 |
| AUC₀_{_{—}}_{Tlast}/Dose (hr*ng/mL)/(mg/kg) | 417 |
| AUC_{INF} (hr*ng/mL) | 5,021 |
| V_{z} (mL/kg) | 5,793 |
| Cl (mL/hr/kg) | 2,390 |

Pharmacokinetic analysis was performed for both IV and IM administration. Table 21 presents the IV PK parameters obtained. The half-life of CBD was rapid, 1.68 h, and the exposure in terms of AUC normalized to the dose was 417 h*ng/ml/mg/kg. The pharmacokinetic analysis following IM administration was performed for the three formulations that were also injected during the 1^{st} study allowing 5 time points for each formulation (2, 24 and 72 h from the previous study and 1 and 3 weeks from current study). The PK analysis of the combined dataset is found in Table 22. F1 and free CBD in PG resulted in the highest AUC. This is in accordance with the finding that for these two groups most of the CBD in the muscles was released by the 3 weeks period (70 and 84% respectively. FIG. 3D) resulting in AUC normalized to the dose values similar to that obtained for the IV injection (388 and 293 h*ng/ml/mg/kg, respectively). The normalized AUC values for Lipo-CBD-HSA 50 mg/ml formulations was lower (167 h*ng/ml/mg/kg) corresponding to the lower % release from muscle obtained for this formulation (68%, respectively, FIG. 3B).

TABLE 22
Pharmacokinetic parameters obtained following IM administration of
CBD formulations injected in this (2nd) study and in the 1st study

| | | | Cmax_D | | AUClast_D |
|---|---|---|---|---|---|
| | Tmax | Cmax | kg*ng/ | AUClast | h*kg*ng/ |
| Group | h | ng/mL | mL/mg | h*ng/mL | mL/mg |
| F1 | 2 | 2,358 | 18.86 | 48,485 | 388 |
| Free CBD in PG | 2 | 852 | 6.82 | 36,576 | 293 |
| Lipo-CBD-HSA | 2 | 702 | 5.62 | 20,852 | 167 |
| 50 mg/ml | | | | | |

### Discussion

The PK profile following IV administration of 12 mg/kg dose was compared to 4 formulations for long acting formulations of CBD administered by the IM route. The plasma profile of the IM injected formulations showed plasma levels that were in the range of the IV plasma profile for as long as at least 3 weeks after injection. The liposomal-CBD-HSA and F1 formulations contained ≥30% of the injected dose compared to 14% left in the muscles of free CBD group. The fact that CBD plasma levels of the IM formulations maintained similar plasma concentrations to that observed for the IV effective dose suggest that these formulations may allow prolonged CBD effect in vivo. The difference in the PK profile allows to selectively design the preferred formulation for a specific desired release profile.

### Example 4—Preparation of DMPC/DPPC-CBD Liposomes

A liposomal formulation of CBD in DMPC:DPPC at a mole ratio of 45:55 was prepared.

### Materials:

1,2-dimyristoyl-sn-glycero-3-phosphocholine (DMPC): Lipoid, Catalog No. 556200 (Lot: 556200-2190329-01)

Dipalmitoylphosphatidylcholine (DPPC): Lipoid Catalog No. 556300 (Lot: 556300-2170149-01)

CBD: THC Pharma

### Results:

The liposome formed comprised a combination of DMPC:DPPC at a mole ratio of 45:55. Lipid phase composition is detailed in Table 23.

TABLE 23
Lipid phase composition of DMPC/DPPC-CBD liposomes

| | | Calculated | Calculated | |
|---|---|---|---|---|
| Material | MW | amount (g) | amount (mmole) | % Molar |
| DMPC | 677.95 | 0.317 | 0.468 | 26.2 |
| DPPC | 734.039 | 0.419 | 0.570 | 31.9 |
| CBD | 314.47 | 0.25 | 0.795 | 44.5 |
| Ethanol, 99% | | 0.69 | | |

Specifically. Histidine (0.155% w/v)-mannitol (4% w/v) buffer (HMB) at pH 6.5 was used as the aqueous phase of this preparation. The lipid and aqueous phases were pre-warmed at 55° C. The lipid phase was then added to the aqueous phase and stirred at 55° C. for 15 min. Ethanol was removed from the formulation following 4 cycles of wash with Histidine mannitol buffer by centrifugation at 4° C.

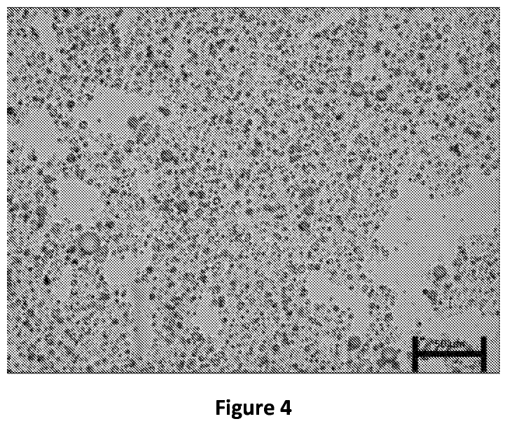

FIG. 4 presents the appearance of the formulation by the microscope (×200 magnification). The mean size of the liposomes was 5.55 μm and total CBD concentration was 25.3 mg/ml.

1. A prolonged release formulation comprising liposomes having a lipid membrane and an intraliposomal aqueous core, wherein said liposome comprises an entrapped cannabinoid and at least one dispersing agent of said cannabinoid, said dispersing agent being other than a cyclodextrin (CD) compound.

2. The prolonged release formulation of claim 1, wherein said liposome comprise entrapped cannabidiol CBD or functional homologue thereof.

3. The prolonged release formulation of claim 1, wherein at least a portion of said cannabinoid is entrapped within said intraliposomal aqueous core.

4. The prolonged release formulation of claim 1, wherein said dispersing agent other than CD is entrapped within said intraliposomal aqueous core.

5. The prolonged release formulation of claim 1, wherein said dispersing agent comprises or is a solubilizer.

6. The prolonged release formulation of claim 5, wherein said solubilizer is selected from Polyoxyethylene (20) sorbitan monooleate and propane-1,2,3-triol:oxirane (1:1).

7. The prolonged release formulation of claim 1, wherein said dispersing agent comprises or is a co-solvent.

8. The prolonged release formulation of claim 7, wherein said co-solvent is selected from polyethylene glycol (PEG) 300, propylene glycol (PG), N,N-dimethylacetamide (DMA) and ethanol.

9. The prolonged release formulation of claim 1, wherein said dispersing agent comprises or is a protein.

10. The prolonged release formulation of claim 9, wherein said protein is a serum protein.

11. The prolonged release formulation of claim 10, wherein said serum protein is selected from human serum albumin and immunoglobulin.

12. The prolonged release formulation of claim 1, comprising in addition to said dispensing agent a cyclodextrin (CD) compound.

13. The prolonged release formulation of claim 12, wherein said CD compound is selected from the group consisting of 2-hydroxypropyl-β-cyclodextrin (HPβCD), 2 hydroxypropyl-γ-cyclodextrin (HPγCD) and Solfobutyl ether (SBE) cyclodextrin.

14. The prolonged release formulation of claim 13, wherein said CD compound is HPβCD.

15. A prolonged release formulation comprising liposomes having a lipid membrane and an intraliposomal aqueous core, the lipid membrane comprises one or more liposome forming lipids, wherein said liposome comprises an entrapped cannabinoid, at least a portion of said cannabinoid being entrapped in said lipid membrane, and wherein said lipid membrane comprises a mole ratio between said cannabinoid and said one or more liposome forming lipids in the range between 1 to 10.

16-23. (canceled)

24. A method of treatment comprising administering to a subject in need of treatment a prolonged release formulation comprising liposomes having a lipid membrane and an intraliposomal aqueous core, wherein said liposome comprises an entrapped cannabinoid and at least one dispersing agent of said cannabinoid, said dispersing agent being other than a cyclodextrin (CD) compound.

25-27. (canceled)

28. A method of treatment comprising administering to a subject in need of treatment a prolonged release formulation comprising liposomes having a lipid membrane and an intraliposomal aqueous core, the lipid membrane comprises one or more liposome forming lipids, wherein said liposome comprises an entrapped cannabinoid, at least a portion of said cannabinoid being entrapped in said lipid membrane, and wherein said lipid membrane comprises a mole ratio between said cannabinoid and said one or more liposome forming lipids in the range between 1 to 10.

29-31. (canceled)

32. The method of claim 24, wherein said administration comprises injection of said prolonged release formulation.

33-36. (canceled)

37. The method of claim 32, wherein said administration comprises injection of said prolonged release formulation.
